(19) United States
(12) Patent Application Publication
(10) Pub. No.: US 2017/0238073 A1
(43) Pub. Date: Aug. 17, 2017

(54) Concrete Maturity System

(71) Applicant: Premiere Concrete Solutions, LLC, Pioneer, OH (US)

(72) Inventors: Douglas Christopher Wittler, Camden, MI (US); Jason Eric May, Defiance, OH (US); John Phillip Gnaedinger, Chesterfield, MO (US)

(73) Assignee: Premiere Concrete Solutions, LLC, Pioneer, OH (US)

(21) Appl. No.: 15/434,221

(22) Filed: Feb. 16, 2017

Related U.S. Application Data
(60) Provisional application No. 62/295,645, filed on Feb. 16, 2016.

(51) Int. Cl. H04Q 9/00; G01N 33/38; H04W 4/00

(52) U.S. Cl. CPC H04Q 9/00; H04W 4/005; G01N 33/383; H04Q 2209/40; H04Q 2209/823

(57) ABSTRACT

Systems and methods for monitoring concrete maturity and other parameters of in-place concrete are disclosed. The systems and methods involve the use of cellular and/or Bluetooth communications. In some embodiments, the systems and methods involve the use of a cellular match-cure box.

### RELATED APPLICATIONS

This application claims priority to U.S. Provisional Application No. 62/295,645, filed under 35 U.S.C. §111(b) on Feb. 16, 2016, the entire disclosure of which is expressly incorporated herein by reference for all purposes.

### BACKGROUND OF THE INVENTION

Concrete is a widely used construction material. Generally, relatively large concrete constructions, called “mass concrete,” tend to show higher temperatures of concrete after placement or deposition than ordinary concrete constructions. This tendency is clearer in the inside of the concrete constructions. In such mass concrete constructions, therefore, strength development of the concrete at early ages becomes very high, and undesirable phenomena which adversely affect the strength of the concrete, such as cracking due to temperature differences between the environment and the mass concrete or within the inside of the mass concrete, can occur. Accordingly, the state of the strength development of concrete should be predicted and controlled appropriately by constructors.

When concrete constructions are being constructed under extraordinary or severe enforcement conditions, such as enforcement in hot and cold seasons, the strength development of concrete is significantly different from that of concrete constructions enforced under normal or mild conditions. Therefore, it is advisable to appropriately predict and control the state of strength development of concrete as stated above.

One method for determining the mechanical strength of a mass of concrete is the “maturity method.” In the maturity method, one maintains a record of the internal temperature history of the concrete mass as it cures. Curing is the strengthening of the concrete through the process of hydration that occurs over a number of days. When concrete stays moist, the moisture allows the chemical reaction between the cementitious materials and water to continue. From the curing temperature history, one can determine the mechanical strength from previously-determined empirical equations. For these equations to be valid, one needs to determine coefficients for these equations that correspond to a given concrete mix design. The coefficients are recalculated for each different mix of concrete. In this sense, the equations are “calibrated” according to the specific mix of concrete to be analyzed. Standards for the maturity method are elaborated in ASTM C1074.

It would be advantageous to provide better systems, devices, and methods for the monitoring of concrete.

### SUMMARY OF THE INVENTION

Provided is a concrete maturity system for the real-time, remote monitoring of in-place concrete temperatures, and estimating in-place concrete compressive and flexural strength using the maturity method. The concrete maturity system includes a node capable of conducting cellular communications, and at least one sensor in communication with the node, where the sensor is configured to collect data of at least one parameter from a location disposed within in-place concrete and communicate the collected data to the node. The node is capable of wirelessly transmitting the collected data to a cloud-based server.

Also provided is a concrete maturity system that includes a match-cure box. The match-cure box houses concrete samples and is capable of maintaining the concrete samples at substantially the same temperature as the in-place concrete in real-time. The match-cure box communicates with the cloud-based server via cellular communications to determine the appropriate temperature at which to keep the concrete samples within the match-cure box. As a result, the concrete samples within the match-cure box are cured at the same time and temperature as the in-place concrete.

Also provided is a method for monitoring concrete, the method including the steps of: (i) inserting sensors into concrete, where the sensors are in communication with a node, the node being capable of conducting cellular communications; (ii) measuring data concerning at least one parameter from the concrete with the sensors; (iii) communicating the measured data to the node; and (iv) transmitting the measured data from the node to a cloud-based server.

### BRIEF DESCRIPTION OF THE DRAWINGS

FIG. 1: Non-limiting illustration of a concrete maturity system for the remote monitoring of in-place concrete.

FIG. 2: Non-limiting illustration of a concrete maturity system utilizing a node having two sensors, and a collector.

FIG. 3: Non-limiting example illustration of a concrete maturity system utilizing multiple pucks.

FIG. 4: Non-limiting example illustration of a concrete maturity system deployed for use in a bridge construction project.

FIGS. 5A-5H: Non-limiting schematics of an example circuit useable in a node in the concrete maturity system.

FIG. 6: Non-limiting illustration of the front of an example circuit board useable in the concrete maturity system.

FIG. 7: Non-limiting illustration of the back of an example circuit board useable in the concrete maturity system.

FIG. 8: Non-limiting illustration of a concrete maturity system that includes a cellular match-cure box.

### DETAILED DESCRIPTION OF THE INVENTION

The term “concrete” is generally used in the construction industry to refer to a mixture of Portland cement or other cementitious or pozzolanic materials, coarse aggregate such as gravel, fine aggregate such as sand, water, and various chemical admixtures which, upon hydration of the cementitious and pozzolanic materials, becomes a hardened mass. “Concrete” may refer to a simple mixture of cement, aggregate, and water. As used herein, the term “concrete” refers to concrete and to formed cement, which is cement and water that hardens into a solid mass upon hydration.

The term “concrete mass” is defined to be a mass or body which is made from concrete, mortar, cement, or the like. This definition is in contrast to the term “concrete form,” which refers to the structures into which concrete is poured to produce a concrete shape that holds its shape upon hardening.

Throughout the present disclosure, the term “system” is used to describe the totality of particular devices, functions, and interfaces described. However, use of the term “system” is not intended to be in any manner limiting. Furthermore, use of the term “system” herein inherently requires at least one physical, tangible structure such as a node or sensor. Therefore, the term “system” should not be construed to refer to mere abstractions devoid of physical structure.

One embodiment of the present disclosure provides a system for measuring, calculating, storing, and displaying concrete maturity and/or strength data within a protected environment and to transfer the data to an intended recipient without risk of loss or alteration of the data at any point. The system of the present disclosure provides for such data to be sent via wireless communications, including via Bluetooth technology or through cell towers.

In accordance with the present disclosure, any number of nodes (also referred to as controllers) are deployed on or around a given concrete placement. Each node is battery-powered, with a battery that generally includes about a three-day reserve amount of power. The nodes have built-in modems and are capable of conducting cellular communications. The nodes can send data to cloud-based servers via cell towers. Therefore, the nodes have virtually unlimited range. The nodes are also capable of storing data on storage devices such as SD cards.

In some embodiments, up to three sensors are attached to each node, such as through sensor ports in the node, via wire leads. The end of each sensor is either embedded directly into the concrete (i.e., is sacrificial and not intended to be retrieved) or encased in a specially-prepared sleeve for later retrieval (if desired). If more than three sensor ports are desired, additional sensor ports can be provided. The sensors are embedded in the concrete at appropriate locations, based on engineering or structural requirements, and the lead wires are routed to the outside of the formwork. In some embodiments, the lead wires are plugged into the wireless nodes. However, it is also possible for the sensors and the nodes to communicate wirelessly, such as through Bluetooth technology. In some embodiments, the sensors are fully retrievable and reusable, which keeps ongoing costs low.

The sensors can be digital temperature probes or other thermocouples, and are preferably reusable. Any number of sensors can be placed in a curing concrete mass to record parameters such as the concrete's temperature and strength, and then send the recorded data to the node placed nearby. In some embodiments, the sensor communicates with the node via Bluetooth technology. Within the concrete mass, the sensors can be arranged to provide real-time data on the critical locations where load- and/or thermal stress-induced tension occurs.

When turned on, in some embodiments, the system automatically commences recording data (such as temperature and strength readings) from the concrete. Examples of possible types of data to be recorded include, but are not limited to: current temperature for each sensor; deployment name, start time, and other jobsite-related details added by the user; temperature history (temperature over time) for each sensor; temperature differential (Δt) between each set of sensors (this aspect is important for certain massive concrete placement applications); current strength of the concrete at each sensor location; maturity over time; additional information, such as battery voltage, cellular signal strength, node ID number, purchase date, warranty, and other node-related details; and archived data for each node. The system may further include an internal sensor located inside the node, to record “ambient” conditions during the deployment.

The nodes can transmit measured data at any desired frequency. In one non-limiting example, the nodes transmit the current temperature readings for all four channels (three sensor ports plus the internal sensor channel) every 10 minutes, wirelessly via the cellular network. In another non-limiting example, measurements are taken every 10 minutes, and transferred to the cloud every 60 minutes. However, the system is customizable such that any desired time schedule of readings and data transfers can be employed.

The data is sent to an internet server (the “cloud”), where it can be processed using any suitable software, including the proprietary software known as Dashboard. At any time, users can log on to a secure website to view all of the live data through a graphical user interface. In some embodiments, the current strength of the concrete at each sensor location can be viewed graphically using a color-changing cylinder, with each color representing the relative strength of the concrete based on the user's required threshold value. For example, the cylinder can be displayed as red if the concrete is less than 90%, yellow if the concrete is between 90% and 100%, and green if the concrete is over 100% of the required strength. This gives users a quick means of assessing strength in the field, without having to dig deeper into the software to observe the actual strength values. However, it is understood that the data can be displayed through the graphical user interface in any manner that is preferable for the particular user and particular application.

The software can be configured to display any or all of the parameters recorded, including real-time temperature history, strength, temperature differentials between all the sensors in the system, and maturity over time. For convenience, all archived data for each node can be made viewable at any time. The data can also be accessed 24 hours a day from any internet browser.

In some embodiments, the maturity over time data is also collected by the system and viewable through the graphical interface. This data aids in understanding how quickly or slowly the concrete is curing. For best durability, concrete should not gain strength too quickly. Therefore, this is a valuable tool for improving concrete quality.

Users can also easily set up email and/or text notifications (i.e., alerts) for critical user-defined thresholds for parameters such as, but not limited to (using x and y as representative of any desired values): high and/or low temperature thresholds (i.e., x°), and then every ±y° thereafter; compressive or flexural strength thresholds (i.e., x PSA or MPa), and then every y PSI/MPa thereafter; temperature differential thresholds (i.e., x°), and then every ±y° thereafter; a low battery; and a damaged, cut, or disconnected sensor lead. The notifications for critical data milestones can be sent to an unlimited number of users. In one example, the interface alerts the user that a particular section of concrete has reached a particular compressive strength, flexural strength, or temperature.

The recorded temperature histories of the concrete can be applied to one or more industry standard formulas, and the concrete strength can be accurately estimated per ASTM C1074. Cloud-based software (such as, but not limited to, the “Dashboard” software) calculates concrete strength, using both widely-accepted maturity formulas: Time-Temperature Factor (TTF, or Nurse-Saul) and Equivalent Age (Arrhenius). Multiple maturity calibration curves can be assigned to a single device, allowing users to monitor different mix designs simultaneously with great simplicity and flexibility.

In addition to, or instead of, wireles sly communicating the recorded data from the node to a cloud-based server, the recorded data can be simultaneously stored on a suitable storage device, such as a mini-SD card located inside the node. This ability provides redundancy and is advantageous in case the system is deployed in an area with poor cellular coverage. In the event the system is deployed in an area with poor cellular coverage, once the node is brought within a viable cellular area, all of the data stored on the SD card can be automatically uploaded to the remote server with no user interaction required.

The present disclosure provides a device, system, and method for cellular-based wireless data transfer directly to a remote cloud-based (internet) server for instant analysis and storage, viewable from any internet-capable device via a secure website. The reusable sensors keep ongoing costs low and encourage widespread use of the system for better coverage and improved information about the construction process. The system and method described herein can be used anywhere concrete is poured or shotcrete is applied, and is particularly beneficial in the following situations: at any jobsite where merely collecting the temperature or maturity data puts a worker at risk; at jobsites that are so large that they require a significant amount of time merely to travel to the monitoring location; tall buildings of any kind; precast plant environments where casting beds are distributed across a large area; and projects requiring live, real-time feedback about the status of the concrete, such as pavement patching jobs on busy interstate highways.

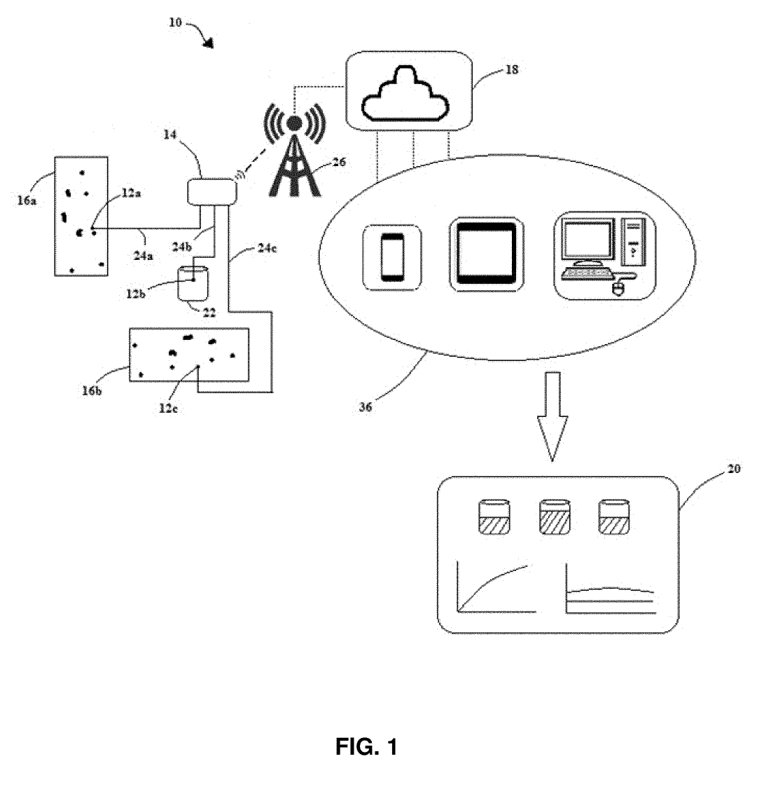

Various example embodiments of the invention will now be described with reference to the drawings. As shown in FIG. 1, an embodiment of the concrete maturity system 10 includes multiple sensors 12a, 12b, 12c attached to a node 14. The sensors 12a, 12b, 12c are disposed in concrete masses 16a, 16b and a test sample 22. The sensors 12a, 12b, 12c are in communication with the node 14 via wire leads 24a, 24b, 24c. The node is configured to transmit recorded data to a cloud-based server 18 via a cell tower 26. The cloud-based server 18 allows any internet-enabled devices 36, such as smart phones, tablets, or computers, to access the recorded data through a secure website. The recorded data is shown through a graphical user interface 20.

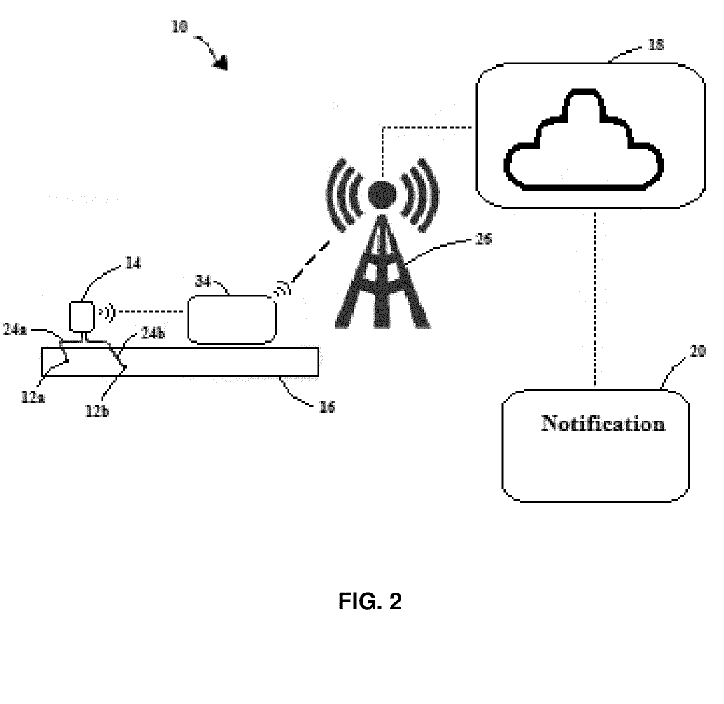

As shown in FIG. 2, an embodiment of the concrete maturity system 10 includes sensors 12a, 12b connected to a node 14 through wire leads 24a, 24b. The sensors 12a, 12b are disposed in a concrete mass 16 so as to gather readings of the temperature and strength of the concrete mass 16. The node 14 is in cellular communication with a collector 34, which is a second node configured to wirelessly transmit the data from one or more nodes such as the node 14. The collector 34, as a node, has a built-in cellular modem that is configured to send the data received from the node 14 to a cloud-based server 18 via a cell tower 26. Users can then access the data in the cloud 18 via a web interface 20. The web interface 20 can also provide additional tools for construction process decision making, such as notification alerts.

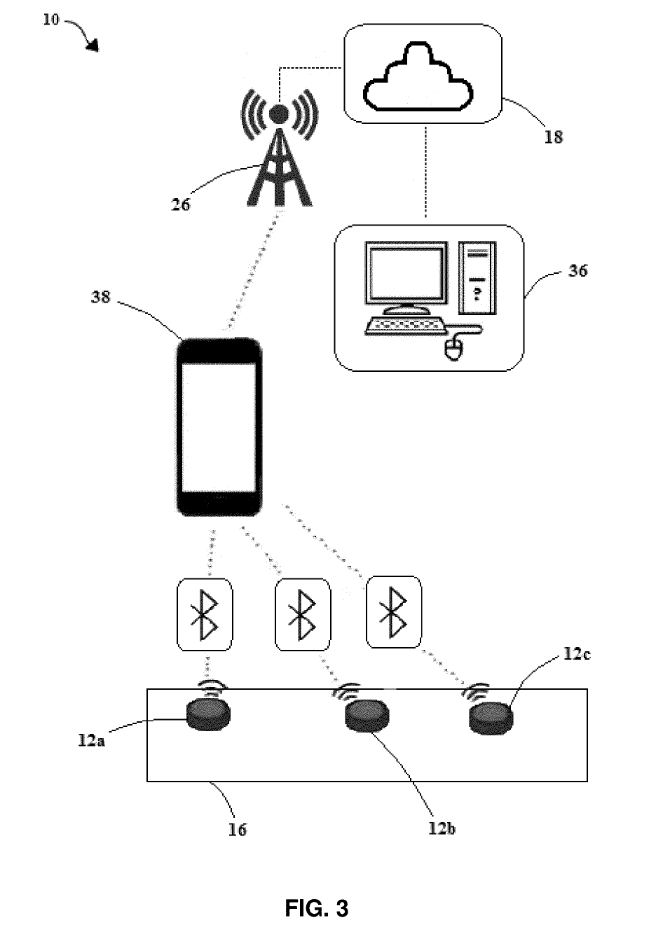

In some embodiments, the sensor 12 is a “puck” sensor embedded in the concrete mass 16. Any number of embedded pucks 12 can be used to record concrete temperatures. As shown in FIG. 3, multiple pucks 12a, 12b, 12c can be configured to record data and transmit the recorded data via Bluetooth to a wireless device 38 within Bluetooth range of the pucks 12a, 12b, 12c. In this manner, a user within Bluetooth range of the puck sensors 12a, 12b, 12c can collect the recorded data on a smart device 38 such as a phone or tablet, as illustrated in FIG. 3. The user merely needs to be within Bluetooth range to collect the data. A device application, such as an iPhone, iPad, or Android app, can simultaneously calculate concrete strength and synchronize the data with the cloud 18. Once in the cloud 18, the data can be visible to any desired subset of users, such as an entire construction team. Thus, in this embodiment, a smart device 38 within Bluetooth range of a sensor 12 can replace the node 14.

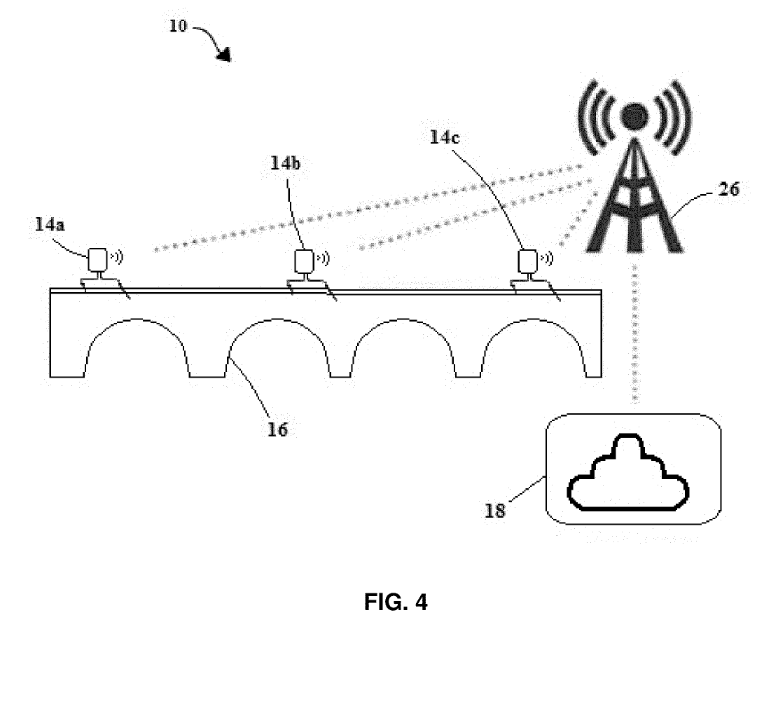

FIG. 4 illustrates an embodiment of the system 10 wherein multiple nodes 14a, 14b, 14c are disposed atop a curing concrete mass 16. In this embodiment, each node 14a, 14b, 14c individually transmits recorded data to the cloud 18. A bridge is depicted in FIG. 4 as the curing concrete mass 16 because the concrete maturity system 10 is particularly advantageous for bridge construction projects, given that bridge construction projects often require a significant amount of time merely to travel to the monitoring location.

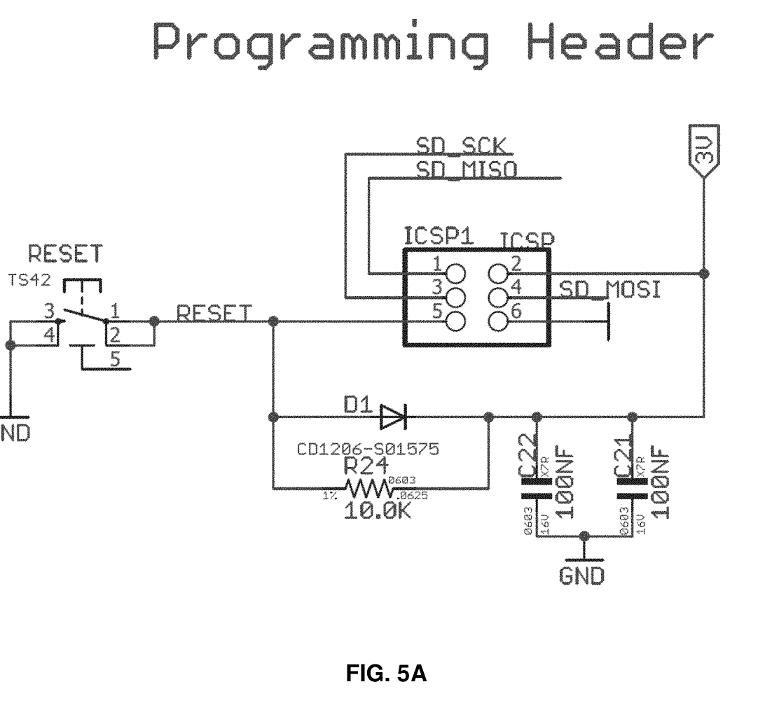

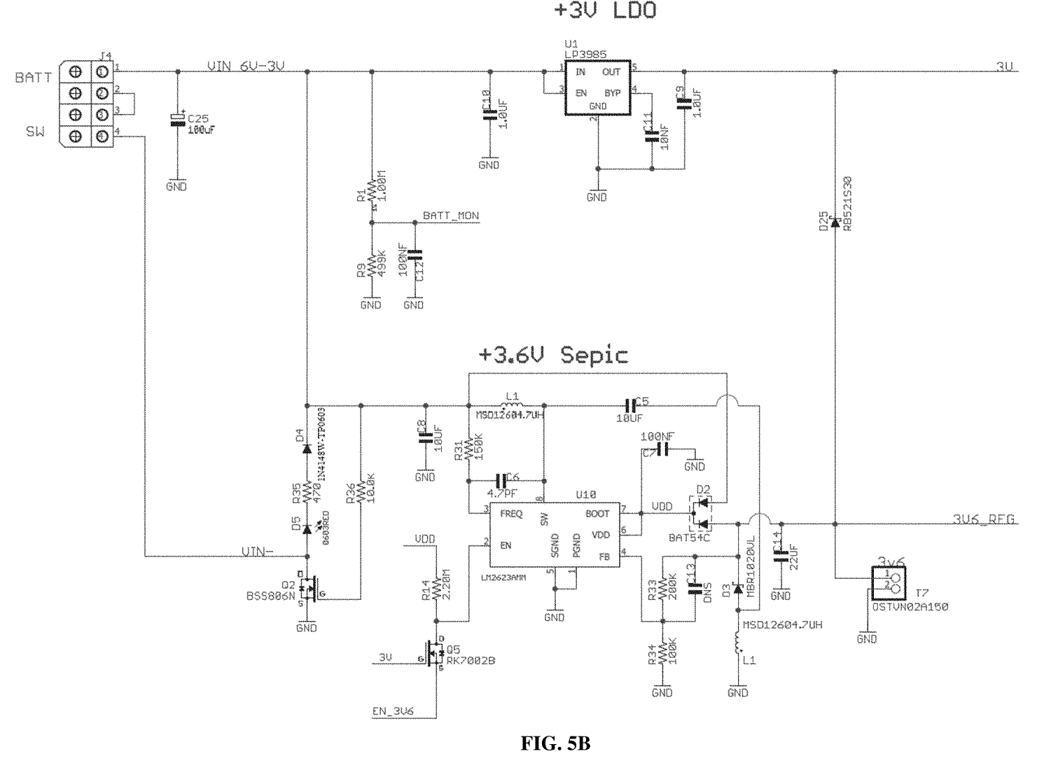

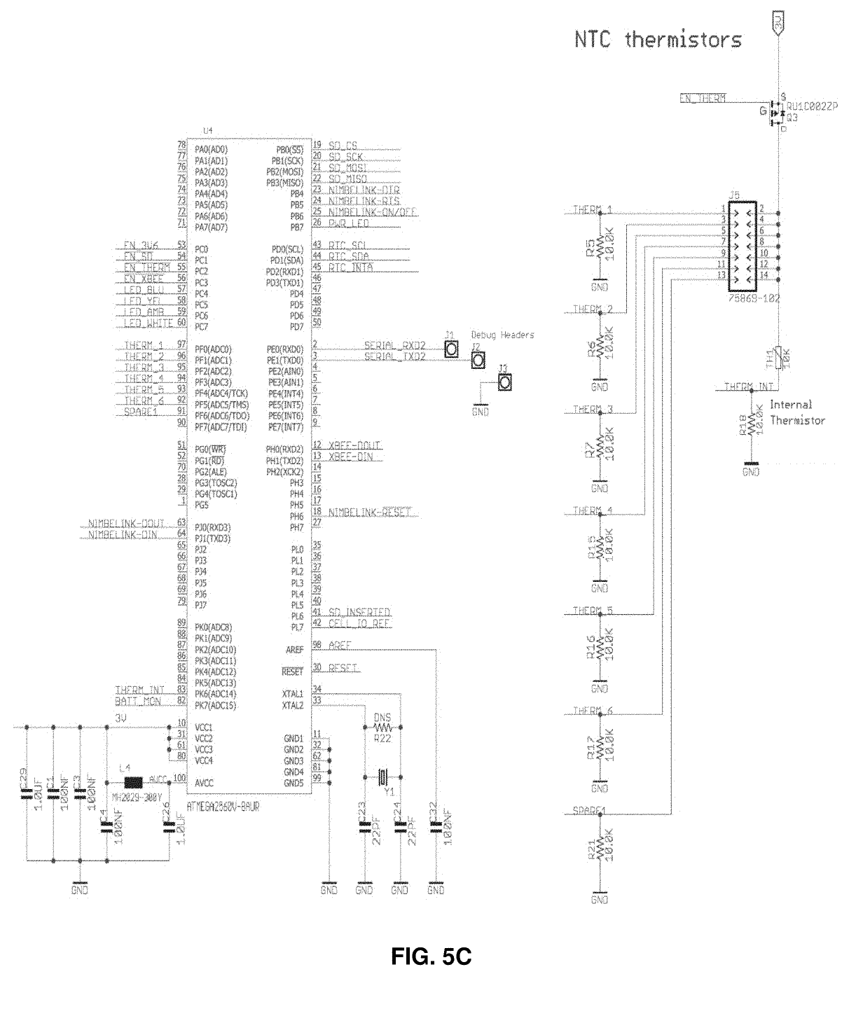

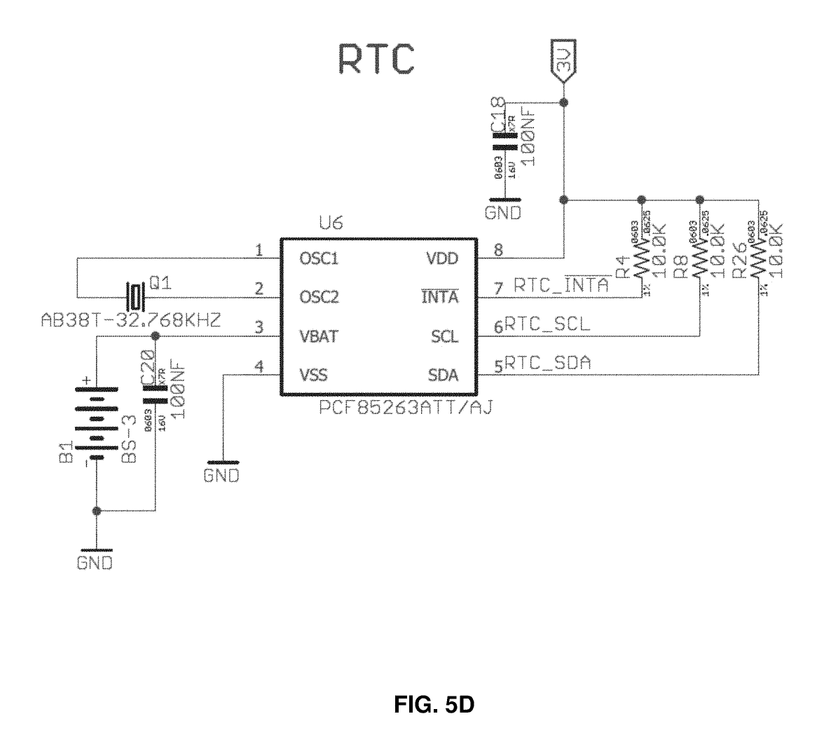

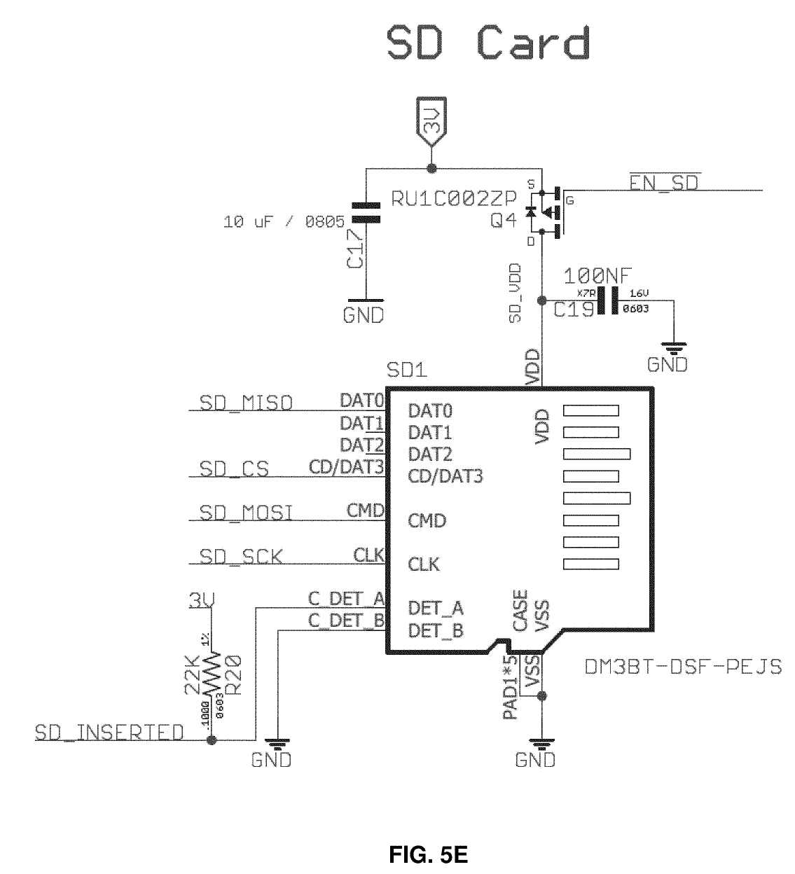

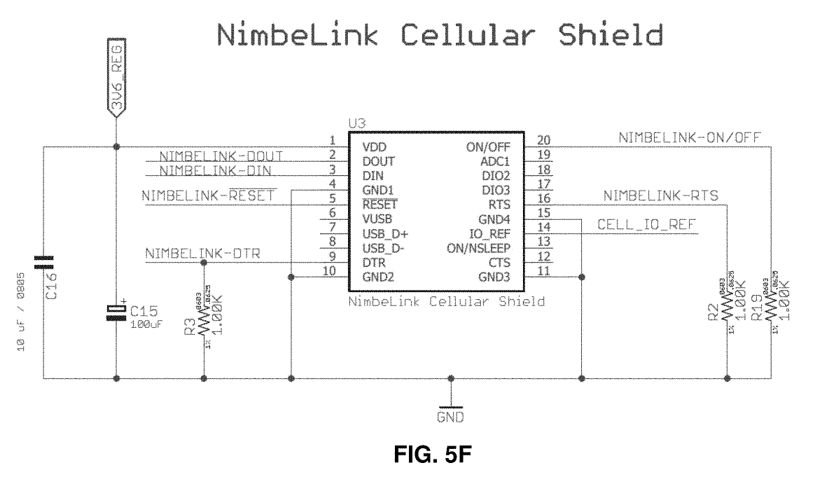

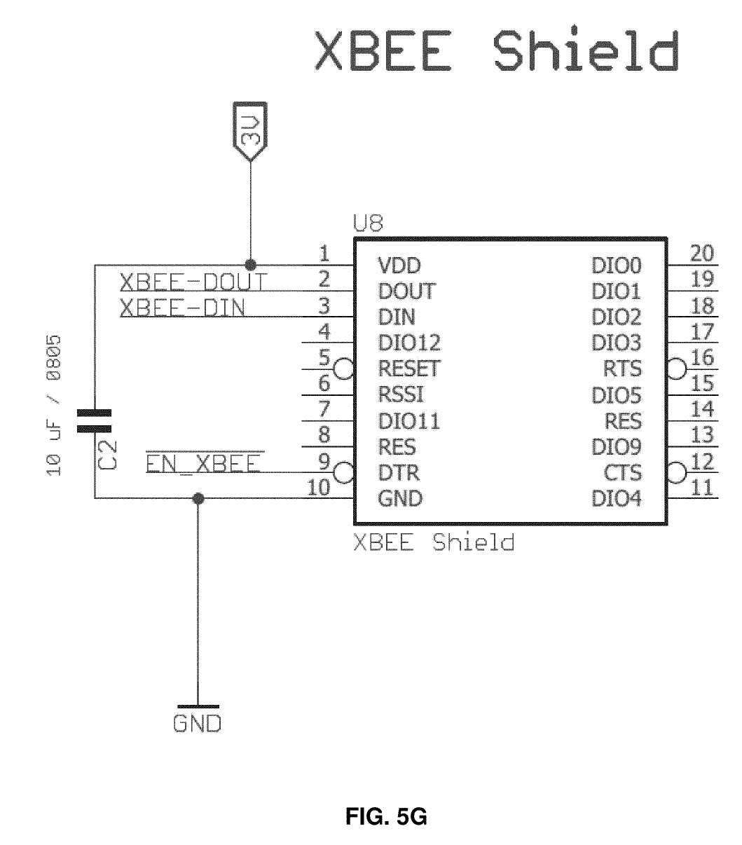

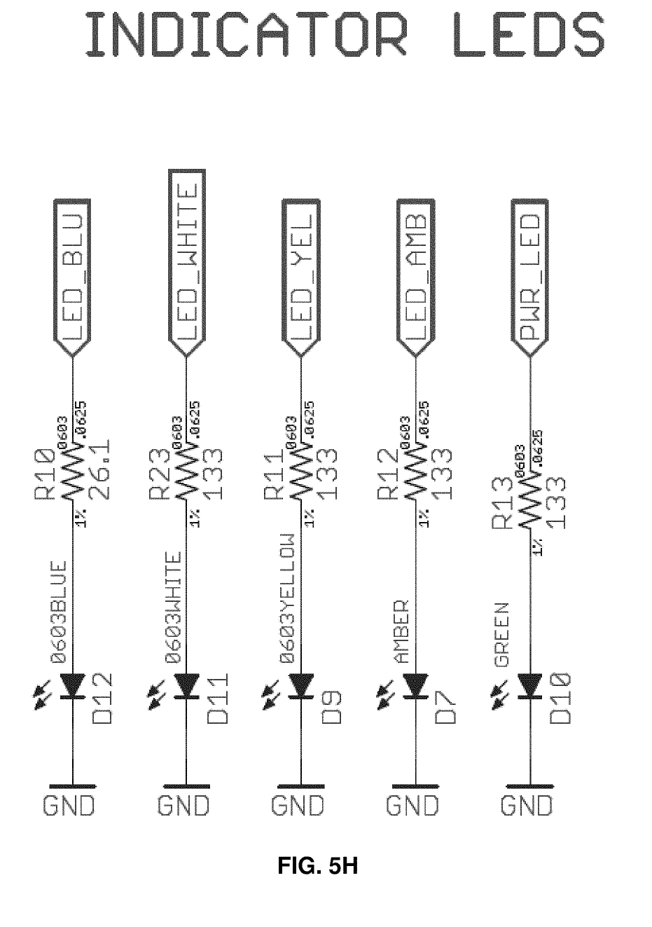

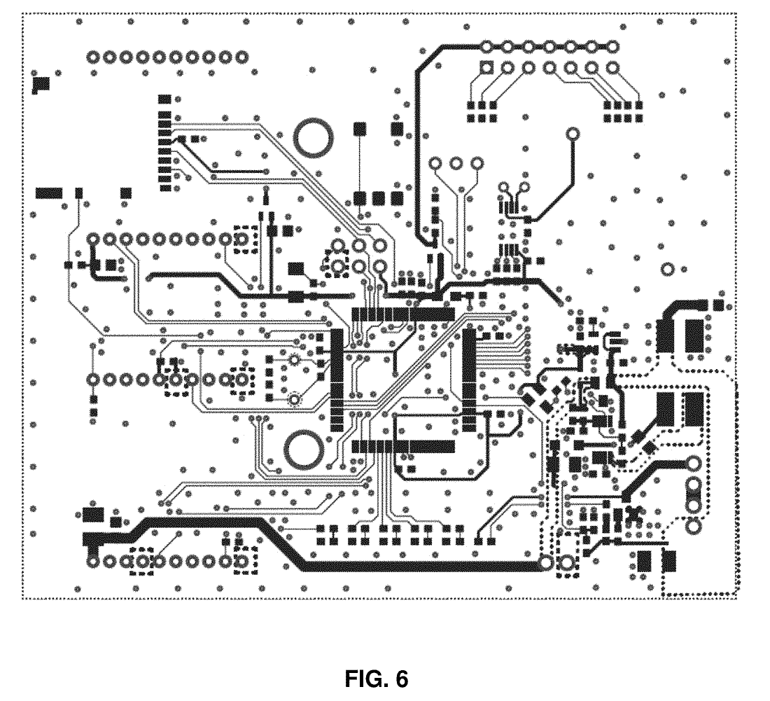

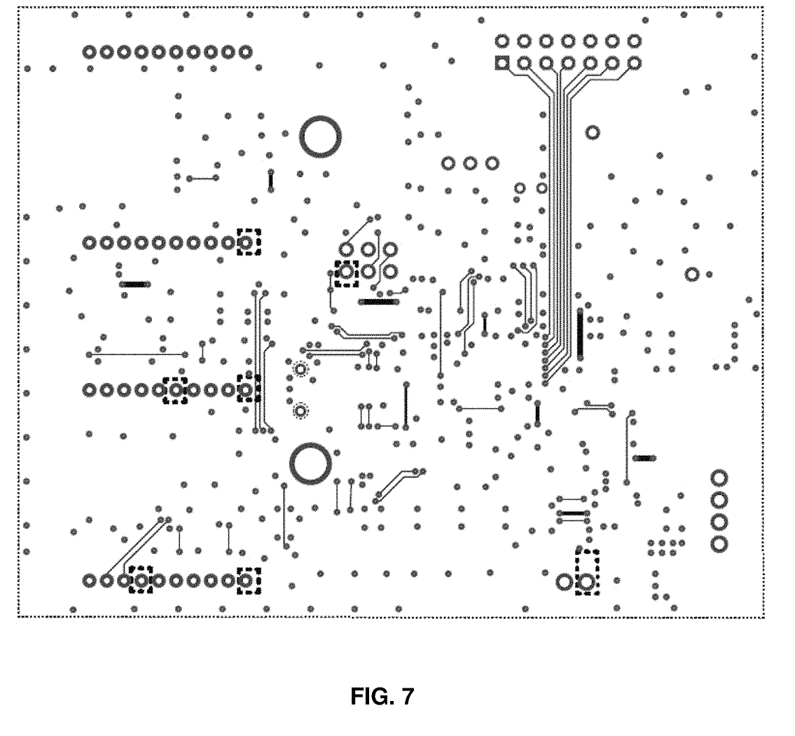

The steps and techniques described herein may be implemented in hardware within one or more application-specific integrated circuits (ASICs), digital signal processors (DSPs), digital signal processing devices (DSPDs), programmable logic devices (PLDs), field programmable gate arrays (FP-GAs), processors, controllers, micro-controllers, microprocessors, or other electronic units designed to perform the functions described. By way of non-limiting examples, FIGS. 5-7 illustrate electronic components usable in the nodes 14 to receive and transmit the data from the sensors 12. FIGS. 5A-5H show a non-limiting example circuit usable in a node 14. FIG. 6 shows a schematic diagram of the front of an example circuit board usable in a node, and FIG. 7 shows a schematic diagram of the back of an example circuit board useable in a node 14. However, it is understood that many other circuits and circuit board layouts which accomplish the same basic functions are possible, and such other circuits and circuit board layouts are entirely encompassed within the scope of the present disclosure.

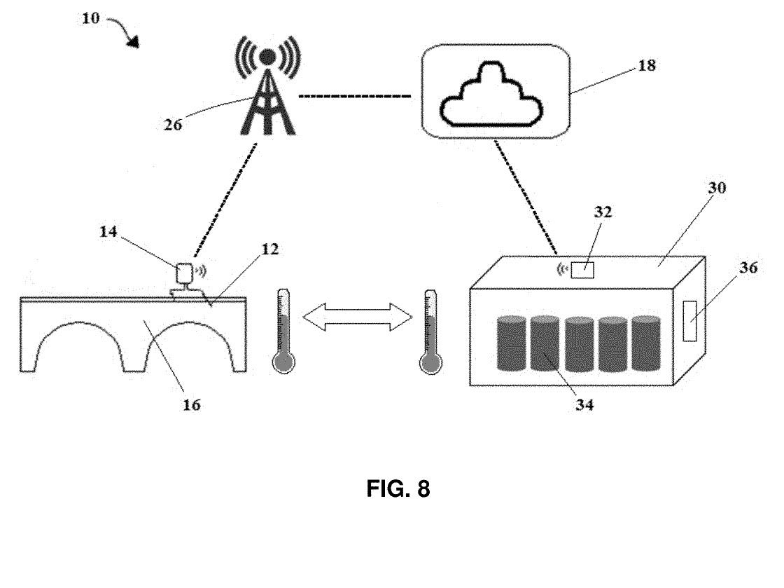

In some embodiments, a concrete maturity system can include a match-cure box 30, as illustrated in FIG. 8. As described above, a node 14 at the site of the curing concrete mass 16 communicates data to the cloud 18. The match-cure box 30 is in cellular communication with the cloud 18 so as to receive the relevant data, such as the temperature data, sent by the node 14. The use of cellular communication allows for the match-cure box 30 to have a simple, portable design by eliminating the need for a large receiver or control unit on the match-cure box 30. Instead, a simple controller 32 within the match-cure box 30 can receive the signal from the cloud 18, and transmit the received signal to a temperature control system 36. The temperature control system 36 is configured to control the temperature of the concrete samples 34 within the match-cure box 30 accordingly.

The match-cure box 30 can be any desired size, with larger match-cure boxes being able to hold more concrete than smaller match-cure boxes, but also being less portable than smaller match-cure boxes. Larger, less portable match-cure boxes are especially useful in labs, while smaller, more portable match-cure boxes are especially useful on construction jobsites. Regardless of its size, the match-cure box 30 provides heating and cooling to manage the temperature of concrete samples inside the match-cure box 30.

The match-cure box 30 is capable of matching, or at least substantially matching, in real time, the temperature profile and strength gain rate of the concrete 16 being cured in another location, such as a jobsite, as monitored via a remote node 14, by associating the remote node 14 with the match-cure box 30 in suitable software, such as the Dashboard software. Concrete samples 34, such as concrete test cylinders, are placed inside the match-cure box 30 at the time of depositing the concrete in the concrete mass 16 at the jobsite. Though cylinders are described and depicted in FIG. 8 for exemplary purposes, it is understood that the concrete samples 34 within the match-cure box 30 may be in any desired shape. The concrete samples 34 may also be of any desired size, so long as they fit within the temperature-controlled area of the match-cure box 30.

The match-cure box 30 retrieves the temperature profile from the cloud-based server 18 over the cellular network, and matches the temperature profile of the remote node 14 to cure the concrete samples 34 at the same rate as the concrete 16 measured by the sensor 12 and transmitted by the remote node 14. It is understood that, though cellular communications are described for exemplary purposes, any means of communicating between the match-cure box 30 and the cloud 18 that allow for the match-cure box 30 to retrieve real-time temperature data transmitted from the node 14 are encompassed within the present disclosure. In fact, the cloud 18 is not a critical component. Rather, it is possible, especially when the match-cure box 30 is used on the jobsite, for the node 14 to be in direct communication with the match-cure box 30, such as via Bluetooth, and thereby bypass the cloud 18.

The match-cure box 30 includes a temperature control system 36 capable of heating and/or cooling the concrete samples 34 within the match-cure box according to the data received from the cloud 18. In one non-limiting example, the match-cure box 30 uses air to maintain the concrete sample temperature. In another non-limiting example, the match-cure box 30 uses water to maintain the temperature of the concrete samples 34. Water is generally more suitable for smaller, more portable embodiments of the match-cure box 30, and air is generally more suitable for larger, less portable embodiments of the match-cure box 30. However, the match-cure box 30 may use other methods of maintaining the temperature. The software effectively controls the temperature control system 36, and integrates identical temperatures and strengths between the concrete 16 and the concrete samples 34.

The match-cure box 30 allows for tests to be conducted on concrete samples 34 at any time knowing that the concrete samples 34 have cured at the same temperature for the same time as the in-place concrete 16 in the field. This is highly advantageous for constructors, as the concrete samples 34 in the match-cure box 30 have substantially the same strength gain as the in-place concrete 16 in the field.

Certain embodiments of the systems, devices, and methods disclosed herein are defined in various examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A concrete maturity system comprising:
a node capable of conducting cellular communications; and
at least one sensor in communication with the node, the sensor being configured to collect data of at least one parameter from a location disposed within in-place concrete and communicate the collected data to the node;
wherein the node is capable of wireles sly transmitting the collected data to a cloud-based server.

2. The system of claim 1, wherein the sensor is embedded within the in-place concrete.

3. The system of claim 1, comprising a plurality of sensors in communication with the node.

4. The system of claim 1, further comprising a web interface for a user to view data transmitted from the node to the cloud-based server.

5. The system of claim 4, wherein the web interface allows a user to set up notifications for critical user-defined thresholds of the at least one parameter.

6. The system of claim 1, comprising a plurality of nodes.

7. The system of claim 6, further comprising a collector configured to receive data transmitted from the plurality of nodes and wireles sly transmit the received data to the cloud-based server.

8. The system of claim 6, wherein each node is configured to individually transmit the collected data to the cloud-based server.

9. The system of claim 1, wherein the node is configured to store the collected data on a storage device.

10. The system of claim 1, further comprising a match-cure box in communication with the cloud-based server, wherein the match-cure box houses one or more samples of concrete, and wherein the match-cure box is configured to control the temperature of the samples of concrete in a manner that corresponds to the collected data.

11. A method of monitoring concrete, the method comprising:
inserting sensors into concrete, wherein the sensors are in communication with a node, the node being capable of conducting cellular communications;
measuring data concerning at least one parameter from the concrete with the sensors;
communicating the measured data to the node; and
transmitting the measured data from the node to a cloud-based server.

12. The method of claim 11, wherein the at least one parameter is selected from the group consisting of temperature, temperature differential between multiple sensors, flexural strength, compressive strength, and concrete maturity.

13. The method of claim 11, wherein the measured data is stored on a storage device.

14. The method of claim 11, further comprising setting an alert to notify a user when the at least one parameter reaches a desired threshold.

15. The method of claim 11, wherein data is measured automatically at a desired frequency.

16. The method of claim 11, wherein the measured data is transmitted from the node to the cloud-based server automatically at a desired frequency.

17. The method of claim 11, comprising a plurality of nodes.

18. The method of claim 17, comprising a plurality of sensors in communication with each of the plurality of nodes.

19. The method of claim 11, further comprising transmitting the measured data from the cloud-based server to a match-cure box over a cellular network, wherein the match-cure box is configured to control the temperature of concrete samples housed within the match-cure box in accordance with the measured data.

20. The method of claim 19, wherein the match-cure box uses air or water to control the temperature of the concrete samples.
